(19) United States
(12) Patent Application Publication
(10) Pub. No.: US 2024/0130720 A1
(43) Pub. Date: Apr. 25, 2024

(54) SURGICAL DEVICE FOR CLAMPING IN TISSUE

(71) Applicant: Eberhard Karls Universität Tübingen Medizinische Fakultät, Tübingen (DE)

(72) Inventor: Sascha Hoffmann, Tübingen (DE)

(21) Appl. No.: 18/525,208

(22) Filed: Nov. 30, 2023

Related U.S. Application Data
(63) Continuation of application No. PCT/EP2022/064952, filed on Jun. 1, 2022.

(30) Foreign Application Priority Data
Jun. 1, 2021 (DE) 10 2021 114 299.7

(51) Int. Cl. A61B 17/00; A61B 90/00

(52) U.S. Cl. CPC A61B 17/00234; A61B 90/08; A61B 2017/00681; A61B 2017/0088; A61B 2017/00982

(57) ABSTRACT

The present invention relates to a surgical device for clamping in tissue, wherein the surgical device comprises a first clamp, a second clamp, and an elongated dimensionally stable connector and its use within a minimally invasive or open surgical treatment for effecting tissue to be self-hold.

### CROSS REFERENCE TO RELATED APPLICATIONS

This application is a continuation of international patent application PCT/EP2022/064952, filed on Jun. 1, 2022 designating the U.S., which international patent application has been published in German language and claims priority from German patent application DE 10 2021 114 299.7, filed on Jun. 2, 2021. The entire contents of these priority applications are incorporated herein by reference.

### BACKGROUND

The present invention relates to a surgical device for clamping in tissue and its use within a minimally invasive or open surgical treatment for self-retaining of tissue.

A distinction is made between open surgery and minimally invasive surgery for the purpose of treatment or diagnostics. Both procedures require a variety of instruments and tools.

In conventional open surgery inside the human body, the surgeon must make a sufficiently large incision to access the area of interest and provide a direct line of sight to the area. However, with the development of endoscopes, surgical tools that can be handled from outside the patient's body, and various imaging techniques such as ultrasound imaging, com-puter tomography, and magnetic resonance imaging, it has become possible to perform surgical procedures through very small incisions or body openings that would be unsuitable in size for traditional open surgery. Such surgical procedures are generally referred to as minimally invasive surgery.

Minimally invasive surgery is very favourable to the patient in cases where it is applicable, as it causes less trauma than open surgery. It also often leads to a reduction in treatment costs and shorter hospital stays. However, conventional minimally invasive surgical devices have several disadvantages. One disadvantage is that the surgeon can easily become fatigued due to the need to manually hold the minimally invasive surgical device during its use. Another disadvantage is that the minimally invasive surgical devices require the surgeon to hold his hands in a very uncomfortable position. In addition, conventional minimally invasive surgical devices can cause angular magnification of incorrect movements. Consequently, the surgeon can only perform an operation using minimally invasive surgical devices with considerably less handling precision and accuracy than when performing an operation using conventional techniques where the surgeon grasps the tool directly. Therefore, minimally invasive surgery is only used for those surgical procedures that require a low degree of handling precision from the surgeon.

A typical surgical device for use in minimally invasive surgery comprises an elongated tube having a first end that is insertable into the patient's body through an incision or opening, and a second end that extends out of the patient's body and is grasped by the surgeon. The first end is provided with a surgical tool, while the second end is provided with a handle or other part which is grasped by the surgeon, and which is mechanically connected to the tool through the center of the tube. The surgeon can actuate the tool by manipulating the handle and can change the position of the tool by changing the orientation of the tube relative to the patient's body.

Generic medical instruments/tools are often used in practice as gripping, holding, and/or cutting tools. For example, the jaws may comprise cutting edges to sever tissue or blunt surfaces to hold severed tissue, for example, or to clamp blood vessels.

Trocars are frequently used today in minimally invasive surgery for inserting surgical implants into body cavities. Such trocars are used, for example, in laparoscopic and thoracoscopic procedures. Instruments and objects can be introduced into the body cavity through the sleeve-shaped trocars leading into the operating space from outside the body. The disadvantage of this is that each instrument has to be placed through a single trocar or a single tube. This means that the number of instruments is always limited by the number of accesses i.e., the number of trocars.

There is still a great need for the development of improved surgical tools for both open surgical procedures and minimally invasive operations.

Against this background, it is an object of the present invention to provide a surgical device which allows a physician to perform a medical procedure in a simplified manner, wherein the above disadvantages shall be overcome.

### SUMMARY

According to the invention, this object is solved by a surgical device for clamping in tissue, wherein the surgical device comprises the following:
a first clamp comprising two clamp-legs and a hinge-like articulated spring holding the clamp-legs together, wherein the first clamp comprises a distal clamping portion config-ured for clamping and holding tissue, and a proximal actuating portion configured for opening and closing the clamping portion of the first clamp,
a second clamp comprising two clamp-legs and a hinge-like articulated spring holding the clamp-legs together, wherein the second clamp comprises a distal clamping portion config-ured to clamp and hold tissue, and a proximal actuating portion configured to open and close the distal clamping portion of the second clamp, and
an elongate dimensionally stable connector for connecting the first and second clamps, wherein the connector is fixed to the proximal actuating portion of the first clamp and to the proximal actuating portion of the second clamp to form a stable distance between the first clamp and the second clamp.

The object is further solved by using the surgical device according to the invention within a minimally invasive or open surgical treatment, preferably within a biopsy/endoscopy for self-hold of tissue.

The object of the invention is completely solved this way.

The present invention provides a surgical device for clamping in tissue, wherein the device is suitable for surgical procedures and preferably for minimally invasive surgical procedures. The procedure may be a diagnostic or therapeutic medical procedure.

With the surgical device according to the invention, a medical tool is provided with which a attending physician is able to clamp tissue during a surgical procedure and to keep it in shape or at a distance during a procedure, for example.

The surgical device according to the invention also offers the advantage that it is independent of a trocar or tube. This means that although it can be guided to the treatment site via a trocar, it can be used there (i.e., at the treatment site) without a trocar, such that several surgical devices according to the invention can be introduced into a body opening through only a single tube.

The device according to the invention or the first and second clamps can be opened by exertion of pressure within the proximal actuating portion of the respective clamp. The exertion of pressure can be effected, for example, by direct finger pressure of the attending surgeon/physician or by another tool, e.g. surgical forceps or endoscopic clamp.

The surgical device according to the invention also provides a medical tool that can also be used in robot-assisting surgery and endoscopy. In such case, medical robotics offers the advantage of high-precision movements and thus robot-assisting handling of the device according to the invention. In this case, the device according to the invention or the first and second clamps, respectively, can be opened by exerting pressure within the proximal actuating portion of the respective clamp. The exertion of pressure can be effected by the robot itself or by a gripping arm of the robot, wherein the robot is controlled, for example, by the attending physician by means of a joystick.

According to the invention, a “clamp” is understood to be a device with which it is possible for an attending physician to clamp tissue or keep the tissue at a distance. The clamps according to the invention or their distal clamping portion are configured such that they preferably do not injure or rupture the tissue during the procedure.

According to the invention, the first as well as the second clamp is configured such that it comprises two clamp-legs, respectively, which are held together by a hinge-like articulated spring. The two clamp-legs form a distal clamping portion and a proximal actuating portion. The distal clamping portion is preferably formed by distal portions of the two clamp-legs. Conversely, the proximal actuating portion is formed by proximal portions of the two clamp-legs. In other words, the respective clamp-legs of the first and second clamps extend from the respective distal clamping portion to the respective actuating portion, wherein they are held together in a hinged manner by the respective spring.

According to one embodiment of the present invention, the first and second clamps can be identical clamps. In this case, preferably the distal clamping portion of the first and second clamps or the distal portions of the two clamp-legs of the first and second clamps can be identically provided. For example, the first and second clamps can be provided as crocodile or alligator clamps, wherein the respective clamp-legs or the distal portions of the respective two clamp-legs of the first and second clamps comprise a row of teeth that can optionally interlock. Alternatively, the first and second clamps can also be provided as surgical forceps, in which two opposing spikes touch each other in the closed state. Other embodiments of the distal clamping portions of the first and second clamps are also possible according to the invention.

Alternatively, the first and second clamps can also be provided in a different form/shape/embodiment. In such case, preferably the distal clamping portion of the first and second clamps or the distal portions of the clamp-legs of the first and second clamps can be differently formed. For example, the first clamp may be formed as a crocodile clamp and the second clamp as surgical forceps, or vice versa.

According to one embodiment of the present invention, the first and second clamps are preferably in a closed handling state. In this state, the respective clamp-legs or the distal portions of the respective clamp-legs of the first or second clamp preferably touch each other in the respective distal clamping portion; that is, in the portion in which the tissue is to be clamped. The closed state of the respective clamp-legs is achieved by the respective springs, which are each relaxed in the closed state of the clamp. Conversely, the respective springs of the respective clamps are compressed i.e., under tension when the clamp-legs or the distal clamp portion are in an open state, i.e., when the respective distal portions of the clamp-legs do not contact each other. Conversely, the proximal portions of the clamp-legs do not contact each other in a closed handling state of the surgical device, whereas the proximal portions of the clamp-legs may contact each other in an open state.

According to one embodiment of the present invention, the spring of the first and/or second clamp is preferably a metallic component that can be elastically deformed. Optionally, the spring may comprise an insulating sheath or coating such that it is non-conductive. Preferably, the spring is in a relaxed handling state, which can be converted into a compressed state, in which the spring is in tension, by exerting pressure on the proximal actuating portion. This offers the advantage that the tissue, which is to be held at a distance, can be securely clamped in the surgical device.

It is further preferred if the spring is a helical spring or helical compression spring or a torsion spring, wherein the torsion spring is particularly preferred.

According to one embodiment of the present invention, the connector is “dimensionally stable”. In this context, “dimensionally stable” means that the connector can be deformed when pressure is applied (and retains/regains its form after the pressure is released); the connector is, thus, not absolutely stiff. Accordingly, the connector according to the invention comprises a limited flexibility.

According to an embodiment of the present invention, it is preferred if the connector is fixed to a proximal portion of one of the two clamp-legs of the respective clamps. Alternatively, the connector can also be fixed in the area of the spring of the respective clamp.

According to one embodiment of the present invention, the fixation of the connector can be achieved, for example, by gluing or welding. Alternatively, the fixation can also be achieved by clamping. For this purpose, the respective clamp or the respective proximal portion of a clamp-leg can comprise a holder into which the connector can be clamped or pushed and then clamped.

According to one embodiment of the present invention, the elongate, dimensionally stable connector can preferably be embodied as a wire, wherein it is preferable that the wire comprises a diameter of from 10 mm to 50 mm, preferably from 15 mm to 40 mm, more preferably from 20 mm to 30 mm. If the device according to the invention is used within a robot-supporting/robot-assisting surgical procedure, the wire of the elongate, dimensionally stable connector may preferably comprise a diameter of 1 mm to 10 mm, preferably 1 mm to 5 mm, more preferably 1 mm to 3 mm.

In such case, it may be preferred that the wire consists of or contains one of the following materials: stainless steel, preferably stainless steel, metal alloys, preferably such made of titanium, tantalum, brass, copper, silver. Furthermore, it may be preferred if the wire is an insulated wire that is non-conductive. A plastic or silicone is preferably used as the insulating material.

According to a preferred embodiment of the invention, the surgical device is configured for and dimensioned for minimally invasive or open surgical insertion into a tissue.

One advantage of this embodiment is that the device according to the invention provides a surgical tool comprising a small diameter, so that the device can be introduced into a small incision or body opening of the patient.

The surgical device according to the present invention is particularly suitable for minimally invasive surgical operations or other medical procedures performed through small incisions in the body wall of a patient. However, in addition to use in minimally invasive surgery, the surgical device according to the present invention can also be used for other medical procedures, such as external surgery or traditional open surgery in which large incisions are made in the body wall. Accordingly, the surgical device according to the invention offers a broad field of application within medicine.

According to another preferred embodiment of the invention, the surgical device is configured for and dimensioned for surgical, robot-assisting insertion into a tissue.

One of the advantages of this embodiment is that the device according to the invention provides a surgical tool that can be significantly smaller than a minimally invasive tool, such that the surgical procedure leads to fewer complications overall and patients recover more quickly than after traditional open surgery, which require a large incision in the abdomen. Accordingly, according to this embodiment, the device according to the invention can comprise a small diameter overall.

According to another preferred embodiment of the invention, the distal clamping portion of the first clamp and the distal clamping portion of the second clamp can be opened by actuating the respective proximal actuating portion.

This embodiment has the advantage that it is possible for an attending or operating physician to open the respective clamp or the respective distal clamping portion by means of a simple manipulation/actuation.

The opening of the respective clamp or the respective distal clamping portion is preferably achieved by the pressure exerted on the respective proximal actuating portion. This pressure can, for example, be exerted on the proximal actuating portion with a clamp or by two fingers of the attending physician. Through the exerted pressure, the respective spring is deformed, such, that the spring is brought from a relaxed state into a compressed state. The spring is, then, under tension. When the tension is released, the distal clamping portion of the respective clamps closes due to the spring effect, whereby tissue can be securely clamped within the respective clamps.

According to another preferred embodiment, the spring of the first and/or second clamp comprises a spring constant of from 0.005 to 0.25 N/mm, preferably from 0.07 to 0.15 N/mm, more preferably from 0.1 to 0.12 N/mm.

According to this embodiment, it is preferred if the spring is a so-called torsion spring.

This embodiment has the advantage that the spring constant or the spring hardness is selected such that only a low pressure is needed to compress the respective spring to open the distal clamping portions. This pressure can be achieved, for example, by means of a surgical or endoscopic clamp, such that the first and/or second clamp can be opened by means of a surgical or endoscopic clamp.

According to another preferred embodiment, the spring of the first and/or second clamp comprises a spring constant of from 0.005 to 0.025 N/mm, preferably from 0.007 to 0.015 N/mm, more preferably from 0.01 to 0.012 N/mm.

This embodiment has the advantage that the spring constant or the spring hardness is selected such that a very low pressure is sufficient/needed to compress the respective spring to open the distal clamping portions. This pressure can be achieved, for example, by means of a grasping arm of a robot which is used within the surgical procedure, such that the first and/or second clamp can be opened in a robot-assisting manner.

According to another preferred embodiment, the distal clamping portion of the first and/or second clamp is formed by distal portions of the clamp-legs, which each comprise a clamping jaw comprising at least one row of teeth.

According to one embodiment of the present invention, the respective two clamping jaws are configured, such, that the tissue can be securely clamped within the clamping jaws without damaging or rupturing the tissue. For this purpose, the clamping jaws are connected to each other via the spring in such a way that the clamping jaws are pressed against each other in a closed handling state.

This embodiment has the advantage that the tissue can be held securely by the clamping jaws or the at least one row of teeth.

According to one embodiment of the present invention, the row of teeth can be embodied as individual teeth arranged one behind the other, which surround a section of the clamping jaws, or as a zig-zag-shaped surface that fills the entire area of the clamping jaw. According to one embodiment of the present invention, either one clamp-leg or each of the two clamp-legs of a clamping jaw may comprise a row of teeth. If both clamp-legs of a clamping jaw comprise a row of teeth, it may be preferred if the teeth of the opposing rows of teeth interlock with one another.

According to another preferred embodiment of the invention, the distal clamping portion of the first and/or second clamp is formed by distal portions of the clamp-legs, which each comprise a terminal spike, wherein the spikes of the clamp-legs point towards each other.

According to one embodiment of the present invention, the spikes are preferably configured, such, that they do not injure the tissue into which they are clamped. For this purpose, it may be preferred if the terminal or distal spikes are rounded. This embodiment has the advantage that the tissue can be held securely by the spikes.

According to another preferred embodiment, the proximal actuating portion of the first and/or second clamp, respectively, is formed by proximal portions of the clamp-legs, which are configured and dimensioned for actuation by a surgical and/or endoscopic clamp.

According to this embodiment, it is advantageous if the proximal actuating portion comprises recesses into which a surgical and/or endoscopic clamp can engage. This embodiment has the advantage that opening and closing of the distal clamping portion of the respective clamps is facilitated and at the same time unintentional slipping of the surgical and/or endoscopic clamp can be prevented.

According to another preferred embodiment, the first clamp is embodied as a crocodile/alligator clamp comprising two clamp-legs with opposing rows of teeth, and the second clamp is embodied as surgical forceps, wherein the surgical forceps comprise two opposing spikes.

It has proven that this embodiment of the clamps is particularly advantageous for clamping tissue, for example to keep tissue at a distance.

According to another preferred embodiment, the surgical device comprises a length (L) of 1.5 to 15 cm, preferably a length of 8 to 10 cm, and even more preferably a length of 8 to 9 cm.

According to this embodiment, the length of the device is measured from the distal clamping portion of the first clamp to the distal clamping portion of the second clamp. It has proven that the specified lengths are particularly advantageous in surgical procedures to keep tissue at a distance and at the same time securely clamp the tissue. Furthermore, the specified lengths offer facilitated handling during the surgical procedure.

According to another preferred embodiment, the first and/or second clamp comprises a length (LK1, LK2) of 0.5 to 4 cm, wherein a length of 2.5 to 3.5 cm is preferred, wherein even more a length of 3 cm is preferred.

According to this embodiment, the length of the first or second clamp, respectively, is measured from the distal clamping portion to the proximal actuating portion. It has been shown that the specified lengths are particularly advantageous for surgical interventions in order to be able to clamp sufficient tissue.

According to another preferred embodiment, the distance between the first clamp and the second clamp defined by the connector is from about 5 mm to about 50 mm, wherein a distance of about 20 mm to 50 mm is preferred.

According to this embodiment, the distance between the first and second clamps or the length of the connector, respectively, is measured from the proximal actuating portion of the first clamp to the proximal actuating portion of the second clamp. It has been shown that the specified lengths are particularly advantageous for surgical procedures to keep tissue at a distance.

According to another preferred embodiment, the surgical device comprises a length (L) of 1.5 to 7 cm, preferably a length of 1.5 to 5.5 cm, and even more preferably a length of 2 to 4 cm.

According to this embodiment, the length of the device is measured from the distal clamping portion of the first clamp to the distal clamping portion of the second clamp. It has been shown that the specified lengths are particularly advantageous for surgical, robot-assisting procedures to keep tissue at a distance and at the same time securely clamp the tissue.

According to another preferred embodiment, the first and/or second clamp comprises a length (LK1, LK2) of 0.5 to 2.5 cm, wherein a length of 1 to 1.5 cm is preferred, wherein even more a length of 1.5 cm is more preferred.

According to this embodiment, the length of the first or second clamp is measured from the distal clamping portion to the proximal actuating portion. It has been proven that the specified lengths are particularly advantageous for surgical, robot-assisting procedures to be able to clamp sufficient tissue.

According to another preferred embodiment, the distance between the first clamp and the second clamp as defined through the connector is from about 5 mm to about 25 mm.

According to this embodiment, the distance between the first and second clamps or the length of the connector is measured from the proximal actuating portion of the first clamp to the proximal actuating portion of the second clamp. It has been shown that the lengths specified above are particularly advantageous for surgical, robot-assisting procedures to keep tissue at a distance.

According to another preferred embodiment, the surgical device, in a handling state in which the first and second clamps are closed, is dimensioned and configured for being received within an introduction sleeve or tube, wherein the introduction sleeve/tube has a diameter of 7 to 15 mm, preferably a diameter of 8 to 10 mm.

This embodiment has the advantage that the surgical device is configured, such, that it can be introduced and removed through an introduction sleeve/tube during a minimally invasive procedure. In order to be guided through the introduction sleeve, the surgical device, thus, preferably comprises a smaller diameter than the introduction sleeve; for example, from 6 to 14 mm, preferably a diameter of 7 to 9 mm, in order not to become clamped or stuck in the introduction sleeve.

According to one embodiment of the present invention, the introduction sleeve may be a tube through which surgical instruments can be introduced into the body cavity during a minimally invasive procedure.

According to another preferred embodiment, the first and/or second clamp and/or the connector is made of or coated with a non-conductive or only slightly electrically conductive material, wherein the material is selected from plastic, silicone, and ceramic.

This embodiment has the advantage that short circuits and unintentional injuries to the tissue during a surgical procedure can be avoided. Such short circuits/injuries can oc-cur when electrically conductive instruments are used during a surgical procedure.

According to another preferred embodiment, the surgical device comprises a removable label.

According to one embodiment of the present invention, the removable label may be a so-called adhesive tag. With the removable label, it is verifiable for an attending physician whether the device according to the invention is still in the body to be treated or has al-ready been removed from the body. For this purpose, the attending physician can remove the label from the device immediately before use within a body cavity and store it safely before he uses the device according to the invention. After completion of the surgical procedure, the attending physician can compare the number of labels with the number of surgical devices, such that no instrument is unintentionally forgotten in the body.

Further advantages are apparent from the figures in the following description of preferred embodiments.

It is to be understood that the features mentioned above and those to be explained below can be used not only in the respective combination described, but also in other combinations or on their own, without departing from the scope of the present invention.

### BRIEF DESCRIPTION OF THE DRAWINGS

In the following, the invention is explained in more detail with reference to the examples and figures given. In the figures:

FIG. 1 a side view of a schematic representation of a first embodiment of the surgical device;

FIG. 2 a front view of a schematic representation of the first embodiment of the surgical device inside an introduction sleeve;

FIG. 3 a side view of a schematic representation of a first embodiment of the first clamp;

FIG. 4 a side view of a schematic representation of a second embodiment of the surgical device with an introduction sleeve and an endoscopic clamp;

FIG. 5 a side view of a schematic representation of a first embodiment of the second clamp; and

FIG. 6 a side view of a schematic representation of a third embodiment of the surgical device with a surgical clamp.

### PREFERRED EMBODIMENTS

Functionally equivalent elements are designated with the same reference signs in all figures, even in different embodiments.

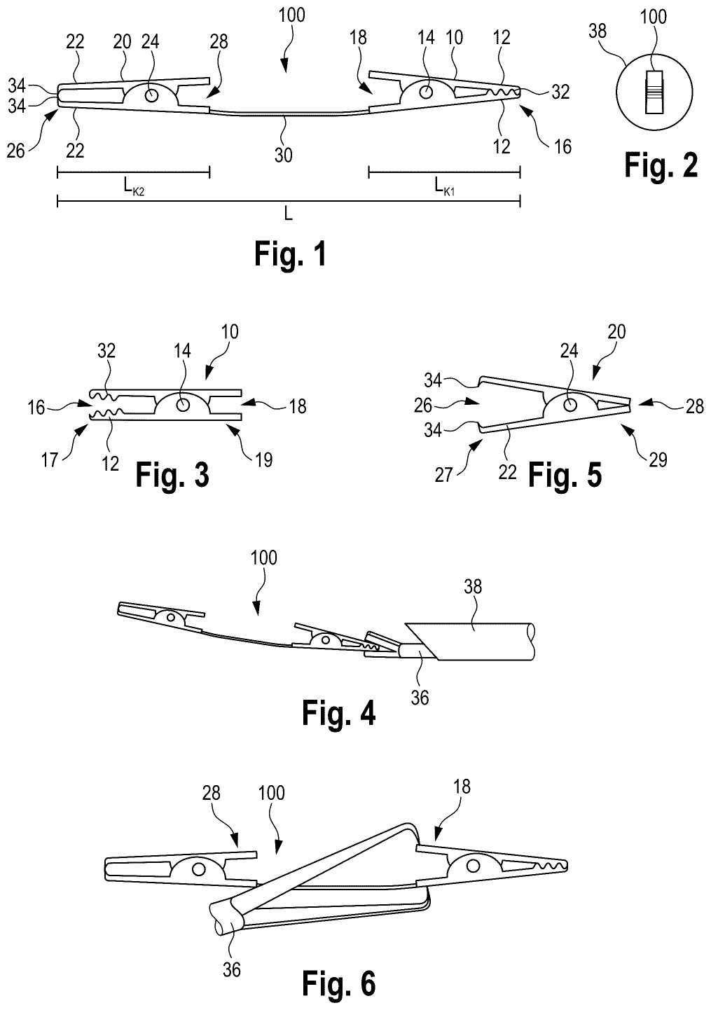

FIG. 1 shows a side view of a schematic representation of a first embodiment of the surgical device 100. The surgical device 100 comprises a first clamp 10, a second clamp 20 and an elongated dimensionally stable connector 30. The two clamps 10 and 20 are in a closed handling state. The length of the surgical device 100 is indicated by L. The length of the first clamp 10 is indicated by LK1, whereas the length of the second clamp 20 is indicated by LK2.

The first clamp 10 is embodied comprising two clamp-legs 12 facing each other, wherein the two clamp-legs 12 are held together by a hinge-like articulated spring 14. The spring 14 as such is not shown in FIG. 1, but only the joint via which the two clamp-legs 12 can be deflected towards each other. The first clamp 10 is shown in a closed handling state. For this purpose, the clamp-legs 12 contact each other in a distal clamping portion 16 or the two distal portions 17 of the clamp-legs 12.

The first clamp 10 comprises a distal clamping portion 16 configured for clamping and holding tissue. The first clamp 10 further comprises a proximal actuating portion 18 for opening and closing the distal clamping portion 16. The distal clamping portion 16 is preferably formed by the distal portion 17 of the clamp-leg 12, whereas the proximal actuating portion 18 is formed by the proximal portion 19 of the clamp-leg 12.

In FIG. 1, the first clamp 10 is embodied as a crocodile clamp 10. The crocodile clamp 10 comprises two opposing rows of teeth 32, which interlock in the closed state. Preferably, the rows of teeth 32 are rounded for that the clamped tissue is not injured/ruptured.

The second clamp 20 is configured to comprise two clamp-legs 22 facing each other, wherein the two clamp-legs 22 are held together by a hinge-like articulated spring 24. The spring 24 as such is not shown in FIG. 1, but only the joint via which the two clamp-legs 22 can be deflected towards each other. The second clamp 20 is shown in a closed handling state. For this purpose, the clamp-legs 22 contact each other in a distal clamping portion 26 or the two distal portions 27 of the clamp-legs 22.

The second clamp 20 comprises a distal clamping portion 26 configured for clamping and holding tissue. The second clamp 20 further comprises a proximal actuating portion 28 for opening and closing the distal clamping portion 26. The distal clamping portion 26 is preferably formed by the distal portion 27 of the clamp-leg 22, whereas the proximal actuating portion 18 is formed by the proximal portion 19 of the clamp-leg 22.

In FIG. 1, the second clamp 20 is embodied or configured as surgical forceps 20. The surgical forceps 20 comprise two opposing spikes 34, which lie on top of each other in the closed state or contact each other at the spike ends. Preferably, the spikes 34 are embodied round so that the clamped tissue is not injured.

In FIG. 1, the first and second clamps 10, 20 are embodied differently; as a crocodile clamp 10 and as surgical forceps 20, respectively. In an embodiment not shown, the two clamps 10, 20 can also be embodied identically respectively.

The elongated, dimensionally stable connector 30 is shown in FIG. 1 as a wire, which is fixed to the proximal actuating portion 18 of the first clamp 10 and to the proximal actuating portion 28 of the second clamp 20 in order to achieve a stable distance between the first and second clamps 10 and 20. In the embodiment shown in FIG. 1, each of the two clamp-legs 12 and 22 of the first and second clamps 10 and 20, respectively, comprises a fixing site at its proximal portion, which is embodied as a cylinder into which the connector can be inserted and fixed. According to one embodiment of the present invention, the connector 30 can also be fixed in a different way, for example in the area of the spring 14, 24.

FIG. 2 shows a front view of a schematic representation of the first embodiment of the surgical device 100 within an introduction sleeve 38. As can be clearly seen in this FIG. 2, the surgical device 100 is preferably configured for and dimensioned for minimally invasive insertion into a tissue, such that the surgical device 100 can be guided through an introduction sleeve 38.

In FIG. 2, the introduction sleeve 38 is shown as simple cylinder. According to one embodiment of the present invention, the introduction sleeve can be a trocar or a tube, via which not only the device 100 according to the invention but also other medical instruments can be guided/introduced.

FIG. 3 shows a side view of a schematic representation of a first embodiment of the first clamp 10. In this embodiment, the first clamp 10 is formed as a crocodile clamp 10; analogous to FIG. 1. According to one embodiment of the present invention, the first clamp 10 can also be formed as a surgical clamp 20, as shown by way of example in FIG. 5.

As depicted in FIG. 3, the clamp 10 is shown to be at least partially opened, such that the two clamp-legs 12 are parallel to each other. In this state, the respective clamp-legs 12 or the distal portions 17 of the respective clamp-legs 12 of the first clamp 10 do not contact each other in the distal clamping portion 16; that is, in the portion in which the tissue is to be clamped.

FIG. 4 shows a side view of a schematic representation of a second embodiment of the surgical device 100 with an introduction sleeve 38 and an endoscopic clamp 36. This figure shows how the surgical device 100 can be guided through an introduction sleeve. In this figure, the introduction sleeve 36 is depicted as a tube 36. The introduction sleeve is preferred in particular for endoscopic treatments. For open surgical treatments, an introduction sleeve may be unnecessary.

For example, the surgical device 100 can be introduced/guided into a body opening of a patient by means of an endoscopic clamp 36. Alternatively, the surgical device may be guided to the treatment site using any other suitable surgical tool. Depending on whether the treatment is an endoscopic or an open surgery one, different introduction-clamps may be advantageous.

FIG. 5 shows a side view of a schematic representation of a first embodiment of the second clamp 20. In this embodiment, the second clamp 20 is formed as/represents a surgical forceps 20; analogous to FIG. 1; analogous to FIG. 1. According to one embodiment of the present invention, the second clamp 20 can also be embodied as a surgical clamp 20, as shown by way of example in FIG. 3. The clamp 20 is shown fully open in FIG. 5, such that the two proximal portions 29 of the clamp-legs 22 are in contact in the proximal actuating portion 18, whereas the distal clamping portion 26 is fully open.

FIG. 6 shows a side view of a schematic representation of a third embodiment of the surgical device 100 with a surgical clamp 36. The mode of operation of the proximal actuating portion 18, which is configured for opening and closing the distal clamping portion 16, is shown. The mode of operation is shown for the first clamp 10 of the surgical device 100. According to one embodiment of the present invention, the second clamp 20 can be actuated anal-ogously through the surgical clamp 36.

By exerting pressure of the surgical clamp 36 on the proximal region 18 of the clamp-legs 12, the two proximal portions 19 of the clamp-legs 12 are pressed against each other, such, that the spring 14 is compressed and the distal portions 17 of the clamp-legs 12 open to clamp the tissue. When the surgical clamp 36 is disengaged from the surgical device, the clamp 10 closes by itself due to the spring action such that the tissue is clamped within the two clamp-legs 12.

In order to be able to ensure secure opening of the clamps 10 and 20, it is advantageous if the proximal actuating portion 18 comprises recesses into which a surgical and/or endoscopic clamp 36 can engage (not shown). This embodiment has the advantage that opening and closing of the distal clamping portion 16 of the respective clamps 10, 20 is facilitated and at the same time unintentional slipping of the surgical and/or endoscopic clamp 36 can be prevented.

What is claimed is:

1. A surgical device for clamping in tissue, wherein the surgical device comprises
a first clamp comprising two clamp-legs and a hinge-like articulated spring holding the clamp-legs together, wherein the first clamp comprises a distal clamping portion config-ured for clamping and holding tissue, and a proximal actuating portion configured for opening and closing the distal clamping portion of the first clamp,
a second clamp comprising two clamp-legs and a hinge-like articulated spring holding the clamp-legs together, wherein the second clamp comprises a distal clamping portion configured for clamping and holding tissue and a proximal actuating portion configured for opening and closing the distal clamping portion of the second clamp, and
an elongated dimensionally stable connector for connecting the first and second clamps, wherein the connector is fixed to the proximal actuating portion of the first clamp and to the proximal actuating portion of the second clamp to form a stable distance between the first clamp and the second clamp.

2. The surgical device of claim 1, wherein the surgical device is configured for and dimensioned for minimally invasive or open surgical insertion into a tissue.

3. The surgical device of claim 1, further being designed such, that by actuation of the respective proximal actuating portion the distal clamping portion of the first clamp and the distal clamping portion of the second clamp can be opened.

4. The surgical device of claim 1, wherein the spring of the first and/or second clamp comprises a spring constant of 0.005 to 0.25 N/mm, preferably of 0.07 to 0.15 N/mm, more preferably of 0.1 to 0.12 N/mm.

5. The surgical device of claim 1, wherein the distal clamping portion of the first and/or second clamp is respectively formed through distal portions of the clamp-legs, which each comprise a clamping jaw comprising at least one row of teeth.

6. The surgical device of claim 1, wherein the distal clamping portion of the first and/or second clamp is respectively formed through distal portions of the clamp-legs, which each comprise a terminal spike, wherein the spikes of the clamp-legs point towards each other.

7. The surgical device of claim 1, wherein the proximal actuating portion of the first and/or second clamp is respectively formed by proximal portions of the clamp-legs, which are configured and dimensioned for actuation by a surgical and/or endoscopic clamp.

8. The surgical device of claim 1, wherein the first clamp is embodied as a crocodile clamp comprising two opposing rows of teeth on the clamp-legs, and wherein the second clamp is provided as surgical forceps, wherein the surgical forceps comprises two opposing spikes.

9. The surgical device of claim 1, wherein the surgical device comprises a length (L) of 1.5 to 15 cm, preferably a length of 8 to 10 cm, and more preferably a length of 8 to 9 cm.

10. The surgical device of claim 1, wherein the first and/or second clamp comprises a length (L_{K1}, L_{K2}) of 0.5 to 4 cm, preferably a length of 2.5 to 3.5 cm, more preferably a length of 3 cm.

11. The surgical device of claim 1, wherein the distance between the first clamp and the second clamp as defined through the connector is from about 5 mm to about 50 mm, preferably from about 20 mm to 50 mm.

12. The surgical device of claim 1, wherein the surgical device, in a handling state in which the first and second clamps are closed, is dimensioned and configured for being accom-modated in an introduction sleeve, wherein the introduction sleeve comprises a diameter of 7 to 15 mm, preferably a diameter of 8 to 10 mm.

13. The surgical device of claim 1, wherein the first and/or second clamp and/or the connector is made of or coated with a material that is not or only slightly electrically conductive, wherein the material is selected from plastic, silicone and ceramic.

14. The surgical device of claim 1, wherein the surgical device comprises a removable label.

15. A surgical treatment system comprising the surgical device of claim 1 and a introduction sleeve.

16. The surgical treatment system of claim 15, being configured for minimally invasive or open surgical treatment, in particular for treatment during a biopsy/endoscopy.

17. The surgical treatment system of claim 15, being configured for robotic-assisted treatment.
